(19) United States
(12) Patent Application Publication
(10) Pub. No.: US 2025/0169957 A1
(43) Pub. Date: May 29, 2025

(54) CARTILAGE REGENERATION SCAFFOLD AND PREPARING METHOD FOR THE SAME

(71) Applicant: NANOBIOSYSTEM CO., LTD., Gwangju (KR)

(72) Inventors: SUNG HO KIM, Gwangju (KR); SANG WON BEOM, Gwangju (KR)

(21) Appl. No.: 18/769,648

(22) Filed: Jul. 11, 2024

(30) Foreign Application Priority Data
Nov. 28, 2023 (KR) 10-2023-0167387

(51) Int. Cl. A61F 2/30; A61L 27/18

(52) U.S. Cl. CPC A61F 2/30756; A61F 2/30771; A61F 2/3094; A61L 27/18; A61F 2002/30766; A61F 2002/30818; A61L 2430/06

(57) ABSTRACT

A scaffold for cartilage regeneration may be manufactured by a scaffold manufacturing apparatus including predetermined molds and structures, such that even when produced in a large area, uniformity in the thickness of a biocompatible polymer sheet and uniformity of a pattern formed on the sheet are excellent, thereby resulting in excellent cartilage regeneration effect and adhesion to the affected site, and a method for manufacturing the cartilage regeneration scaffold.

### CROSS-REFERENCE TO RELATED APPLICATION AND CLAIM OF PRIORITY

This application claims the benefit under 35 USC § 119 of Korean Patent Application No. 10-2023-0167387, filed on Nov. 28, 2023, in the Korean Intellectual Property Office, the entire disclosure of which is incorporated herein by reference for all purposes.

### BACKGROUND

### 1. Field of the Invention

The present invention relates to a scaffold for cartilage regeneration and a method for manufacturing the cartilage regeneration scaffold.

### 2. Description of the Related Art

Cartilage is a bone tissue composed of chondrocytes and a cartilage matrix surrounding these cells. The chondrocytes function to synthesize and secrete the cartilage matrix within the cartilage. The cartilage matrix serves to add elasticity to the cartilage. Unlike other tissues, since blood vessels or nerves are not dispersed in the cartilage, once the cartilage is damaged, it is difficult to regenerate. Due to such insufficient self-repair ability of the cartilage, surgical treatment such as microfracture surgery to treat the damaged cartilage is required.

Microfracture surgery is a surgical procedure aimed at regeneration of the damaged cartilage, and is a surgical technique using the principle that when the cartilage is damaged, fine fractures are created in the exposed bone, and if the subchondral bone is damaged, bone marrow components including bone marrow stem cells leak out, and these cells differentiate to form cartilage.

Most of the cartilage created by the microfracture surgery is fibrocartilage, not hyaline cartilage. Fibrocartilage is rich in type 1 collagen and has a low proteoglycan content, thus it has a low ability to withstand wear. Therefore, when the damaged cartilage tissue is regenerated into fibrocartilage, improvement in symptoms is exhibited by 60 to 70% for up to 2 years after surgery, but after that, structural breakdown occurs to cause worsening symptoms sometimes. In addition, it is known that, as the defect site is increased, the symptoms become more severe. The microfracture therapy method has a limitation in treatment for a wide range of cartilage defects and has a disadvantage that fibrocartilage with weak mechanical properties is regenerated. Therefore, it is necessary to promote cartilage regeneration and improve maturity during a cartilage regeneration process by implanting a cartilage regeneration scaffold into the cartilage defect site along with the microfracture therapy.

The cartilage regeneration scaffold should have uniform thickness and pattern so that chondrocytes can grow densely and consistently. However, existing therapeutic methods have a limitation in uniformizing the thickness and pattern of the cartilage regeneration scaffold. In particular, when manufacturing a cartilage regeneration scaffold to have a predetermined area or more, the unevenness of thickness and pattern is increased.

### SUMMARY

An aspect of the present invention is to provide a cartilage regeneration scaffold having uniform thickness and pattern, and a method for manufacturing the cartilage regeneration scaffold.

Another aspect of the present invention is to provide a cartilage regeneration scaffold that can be attached to a cartilage defect site with high adhesion, and a method for manufacturing the cartilage regeneration scaffold.

To achieve one or more of the above aspects, the following technical solutions are adopted in the present invention.
1. A cartilage regeneration scaffold which is made of a biocompatible polymer, and has a pattern for cartilage regeneration formed on single or both sides, wherein the pattern includes repeating crests and troughs, and an area of the pattern, which satisfies a uniformity (U) of Equation 1 below, is 95% or more of a total area of the pattern:

$$U = \vert {H}_{P 1} - {H}_{P 2} \vert \leq 0.1 {H}_{n} \qquad [ Equation 1 ]$$

(in Equation 1 above, H_{P1} refers to a height at P1, H_{P2} refers to a height at P2, and Hₙ refers to a height difference between the crest and the trough in a normal pattern).
2. The cartilage regeneration scaffold according to the above 1, wherein both P1 and P2 are points located on the crest or on the trough.
3. The cartilage regeneration scaffold according to the above 1, wherein the area which satisfies a uniformity of Equation 1 is 99% or more of the total area of the pattern.
4. The cartilage regeneration scaffold according to the above 1, wherein the scaffold has a thickness of 30 to 100 μm.
5. The cartilage regeneration scaffold according to the above 1, wherein the biocompatible polymer is any one selected from the group consisting of polycaprolactone, polylactide-co-glycolide, polyethylene glycol, polyethylene oxide, polylactic acid and polyglycolic acid.
6. The cartilage regeneration scaffold according to the above 1, wherein the biocompatible polymer is polylactide-co-glycolide including 65 to 85 mol % of lactide and 15 to 35 mol % of glycolide.
7. The cartilage regeneration scaffold according to the above 1, wherein the pattern has a straight line or curved line shape in which the crests and the troughs are repeated side by side.
8. The cartilage regeneration scaffold according to the above 1, wherein the single or both sides is/are applied with any one selected from the group consisting of collagens, growth factors, stem cells, exosomes and therapeutic drugs.
9. The cartilage regeneration scaffold according to the above 1, wherein fibrin is applied to an edge of the scaffold.
10. A method for manufacturing the cartilage regeneration scaffold according to any one of the above 1 to 9, the method including: applying a polymer solution containing a biocompatible polymer to a polyurethane acrylate mold to prepare a sheet-like semi-cured product; applying pressure to the semi-cured product with a polydimethylsiloxane mold to prepare a patterned semi-cured product; and drying the patterned semi-cured product.
11. The method according to the above 10, wherein the patterned semi-cured product is prepared by placing the semi-cured product between the polyurethane acrylate mold and the polydimethylsiloxane mold, and then applying pressure thereto with upper and lower plates.
12. The method according to the above 10, wherein the polyurethane acrylate mold or the polydimethylsiloxane mold has concavo-convex portions formed thereon, for forming the pattern.
13. The method according to the above 11, wherein the upper plate includes a pressing rod and a guide rod.
14. The method according to the above 10, wherein the pressure is 0.40 to 0.70 Pa.

The cartilage regeneration scaffold of the present invention is excellent in terms of cartilage regeneration effect.

The cartilage regeneration scaffold of the present invention is excellent in terms of adhesion.

The cartilage regeneration scaffold of the present invention is excellent in terms of workability.

### BRIEF DESCRIPTION OF THE DRAWINGS

The above and other aspects, features and other advantages of the present invention will be more clearly understood from the following detailed description taken in conjunction with the accompanying drawings, in which:

FIG. 1 is a conceptual diagram illustrating a manufacturing process of a cartilage regeneration scaffold according to the present invention;

FIG. 2 is a view schematically illustrating a method for measuring uniformity (U) in Equation 1;

FIG. 3 is a perspective view illustrating an apparatus for manufacturing a cartilage regeneration scaffold (“cartilage regeneration scaffold manufacturing apparatus”) according to an embodiment of the present invention;

FIG. 4 is a front view illustrating the cartilage regeneration scaffold manufacturing apparatus according to an embodiment of the present invention;

FIG. 5 is a top view illustrating the cartilage regeneration scaffold manufacturing apparatus according to an embodiment of the present invention;

FIG. 6 is a cross-sectional view taken on A1-A2 in FIG. 5;

FIG. 7 is a block diagram illustrating the cartilage regeneration scaffold manufacturing apparatus according to an embodiment of the present invention;

FIG. 8 is a plan view illustrating a shape of a pressing plate of a pressing unit according to an embodiment of the present invention;

FIG. 9 is a plan view illustrating another shape of the pressing plate of the pressing unit according to an embodiment of the present invention;

FIG. 10 is a plan view illustrating another shape of the pressing plate of the pressing unit according to an embodiment of the present invention;

FIG. 11 is a flowchart illustrating a general sequence of a method for manufacturing a cartilage regeneration scaffold (“cartilage regeneration scaffold manufacturing method”) using the cartilage regeneration scaffold manufacturing apparatus according to an embodiment of the present invention;

FIGS. 12 to 14 are flowcharts illustrating detailed sequences of the cartilage regeneration scaffold manufacturing method according to each step in FIG. 11;

FIGS. 15A to 20 are process diagrams illustrating the cartilage regeneration scaffold manufacturing method using the cartilage regeneration scaffold manufacturing apparatus according to an embodiment of the present invention;

FIG. 21 is a photograph of a PLGA large-area sheet according to an embodiment of the present invention; and

FIG. 22 is a photograph measuring a thickness of the large-area PLGA sheet according to an embodiment of the present invention.

FIG. 23 is a photograph measuring a pattern between troughs and crests of large-area PLGA sheet according to an embodiment of the present invention.

### DETAILED DESCRIPTION

The present invention provides a scaffold for cartilage regeneration and a method for manufacturing the cartilage regeneration scaffold.

The present invention provides a scaffold for cartilage regeneration, which is manufactured by a scaffold manufacturing apparatus including predetermined molds and structures, such that even when produced in a large area, uniformity in the thickness of a biocompatible polymer sheet and uniformity of a pattern formed on the sheet are excellent, thereby resulting in excellent cartilage regeneration effect and adhesion to the affected site, and a method for manufacturing the cartilage regeneration scaffold.

The present invention provides a cartilage regeneration scaffold which is made of a biocompatible polymer and has a pattern for chondrocyte growth formed on single or both sides thereof, wherein the pattern includes repeating crests and troughs, and an area of the pattern, which satisfies a uniformity (U) of Equation 1 below, is 95% or more of a total area of the pattern:

$$U = \vert {H}_{P 1} - {H}_{P 2} \vert \leq 0.1 {H}_{n} \qquad [ Equation 1 ]$$

(in Equation 1 above, H_{P1} refers to a height at P1, H_{P2} refers to a height at P2, and Hₙ refers to a height difference between the crest and the trough in a normal pattern).

The cartilage regeneration scaffold of the present invention has high uniformity and appropriate elongation and tensile strength as a cartilage regeneration scaffold, despite its thin thickness, thereby resulting in excellent cartilage regeneration effect and excellent adhesion to the affected site.

The cartilage regeneration scaffold of the present invention is made of a biocompatible polymer.

The biocompatible polymer is not limited to those made of specific materials. For example, the biocompatible polymer may be any one selected from the group consisting of polylactide-co-glycolide (PLGA), polycaprolactone (PCL), polyethylene glycol (PEG), polyethylene oxide (PEO), polylactic acid (PLA) and polyglycolic acid (PGA).

It is preferable that the cartilage regeneration scaffold is made of polylactide-co-glycolide or polycaprolactone in terms of the uniformity and physical properties (such as an elongation rate, tensile strength, etc.). It is more preferable that the cartilage regeneration scaffold is made of PLGA including a lactide (LA) monomer and a glycolide (GA) monomer in a molar ratio of 65-85 to 35-15 in order to achieve the aspects of the present invention.

The biocompatible polymer of the present invention has a pattern for chondrocyte growth formed on single or both sides thereof.

When attaching the cartilage regeneration scaffold to the affected site, chondrocytes existing around the scaffold grow along the troughs of the pattern to induce regeneration of the damaged cartilage.

The pattern may be formed on a single side or both sides of the scaffold. To facilitate distinguish between upper and lower surfaces of the cartilage regeneration scaffold, the pattern may be formed only on the upper surface or lower surface. In addition, if the cartilage regeneration scaffold has a small size, the patterns may be formed on both sides thereof so that there is no distinction between the upper and lower surfaces.

The pattern is not limited to a specific shape as long as it has repeating crests and troughs. The pattern may have, for example, a straight line or curved line shape in which crests and troughs are repeated side by side. The pattern may be formed in a shape such as a straight line parallel to any one edge of the cartilage regeneration scaffold, an oblique line forming a predetermined angle with any one edge, a wavy curve and the like.

The crest refers to a relatively protruding portion, and the trough refers to a relatively depressed portion which exists between the crests. Chondrocytes grow along the trough between the crests.

A width between the crests may be designed variously such as 600 to 1000 nm, 700 to 1000 nm, 800 to 1000 nm, 600 to 900 nm, 600 to 800 nm, 600 to 700 nm and the like.

A width between the troughs may also be designed to be the same as the width between the crests.

A height difference between the crest and the trough (a height of the crest based on the trough) may be designed variously such as 600 to 1000 nm, 700 to 1000 nm, 800 to 1000 nm, 600 to 900 nm, 600 to 800 nm, 600 to 700 nm and the like. A distance between the crests, a distance between the troughs, and the height difference between the crest and the trough of the pattern may be the same as or different from each other. If these are different from each other, differences therebetween may be 100 nm or less, 90 nm or less, 80 nm or less, 70 nm or less, 60 nm or less, or 50 nm or less, respectively.

For example, the distance between the crests, the distance between the troughs, and the height difference between the crest and the trough may be the same as each other such as 800 nm, 700 nm or 600 nm, respectively. In addition, the distance between the crests, and the distance between troughs may be 800 nm, and the height difference between the crest and the trough may be 750 nm or 850 nm. Further, the distance between the crests and the distance between the troughs may be 700 nm, and the height difference between the crest and the trough may be 650 nm or 750 nm.

The distance between the crests, the distance between the troughs, and the height difference between the crest and the trough of the pattern may be changed by adjusting the size and spacing of the pattern formed on the mold used to manufacture the cartilage regeneration scaffold.

The area of the pattern, which satisfies the uniformity (U) of Equation 1 below, is 95% or more, 96% or more, 97% or more, 98% or more, 99% or more, 99.5% or more, or 99.9% or more of the total area of the pattern.

$$U = \vert {H}_{P 1} - {H}_{P 2} \vert \leq 0.1 {H}_{n} \qquad [ Equation 1 ]$$

In Equation 1 above, H_{P1} refers to a height at P1, H_{P2} refers to a height at P2, and Hₙ refers to a height difference between the crest and the trough of the normal pattern.

Both P1 and P2 are arbitrary points on the crest of the pattern or are arbitrary points on the trough of the pattern.

For example, P1 and P2 may be points on the same crest. P1 may be a point on any crest, and P2 may be a point on a crest adjacent to the crest, that is, a point on another crest that exists across one through existing on the left or right of the crest. In addition, P1 may be a point on any crest, and P2 may be a point on a crest spaced apart from the crest, that is, a point on another crest that exists across a plurality of troughs existing on the left or right of the crest.

H_{P1} is a height at P1. The height at P1 means a height from the trough to a point P1 which is any point existing on the crest. If the crest is formed at the intended height, H_{P1} is equal to the height of a pattern formed on the mold, and is equal to Hₙ, which is the height difference between the crest and the trough of the normal pattern. If the pattern is poorly formed, H_{P1} will be different from the height of the pattern formed on the mold.

H_{P2} refers to a height at P2, which is the same as H_{P1}.

The cartilage regeneration scaffold of the present invention has a height difference between P1 and P2 of 10% or less, 9% or less, 8% or less, 7% or less, 6% or less, 5% or less, 4% or less, 3% or less, 2% or less, 1% or less, 0.5% or less, or 0.1% or less of the height difference (Hₙ) between the crest and the trough of the normal pattern.

If the cartilage regeneration scaffold has excellent uniformity in the thickness and pattern, cell proliferation of chondrocytes is smoothly performed along the uniform pattern, thereby showing an excellent cartilage regeneration effect. If the pattern is not uniform and a partially damaged pattern exists, cell proliferation may be stopped in the portion where the pattern is damaged.

When P1 and P2 are points on the same crest, when measuring the uniformity of the pattern, optionally, a height difference between P3 and P4, which are arbitrary points on the trough of the pattern, may be additionally considered.

P3 and P4 may be points on the same trough. P3 may be a point on any trough, and P4 may be a point on a trough adjacent to the trough, that is, a point on another trough that exists across one crest existing on the left or right of the trough. In addition, P3 may be a point on any trough, and P4 may be a point on a trough spaced apart from the trough, that is, a point on another trough that exists across a plurality of crests existing on the left or right of the trough.

When P1 and P2 are arbitrary points on the crest and P3 and P4 are arbitrary points on the trough, an area which satisfies U₁=|H_{P1}−H_{P2}|≤0.1Hₙ and U₂=|H_{P3}−H_{P4}|≤0.1Hₙ (H_{P3} refers to a height at P3, H_{P4} refers to a height at P4 and Hₙ refers to a height difference between the crest and the trough of the normal pattern) may be 95% or more, 96% or more, 97% or more, 98% or more, 99% or more, 99.5% or more, or 99.9% or more of the total area of the pattern.

The cartilage regeneration scaffold may have a thickness of 30 to 100 μm, preferably 40 to 70 μm, and more preferably 50 to 60 μm. If the thickness is thinner than 30 μm, the mechanical strength (tensile strength and compressive strength) is low, such that the scaffold or a nanopattern formed on the scaffold may be easily damaged. If the thickness is thicker than 100 μm, the adhesion is decreased, thereby making it difficult for the scaffold to remain attached to the cartilage for a sufficient period of time required for cartilage regeneration.

The cartilage regeneration scaffold may have a tensile strength of 3 to 8 MPa, and preferably 4 to 6 MPa. The cartilage regeneration scaffold may have an elongation rate of 4 to 10%, and preferably 5 to 7%. If the tensile strength is less than 3 MPa or the elongation rate exceeds 10%, the scaffold is easily deformed, and it is difficult to handle, as well as patients may feel uncomfortable after the procedure. If the tensile strength exceeds 8 MPa or the elongation is less than 4%, the scaffold is vulnerable to breakage and has insufficient flexibility, such that a problem of being not properly attached to the cartilage may occur.

The cartilage regeneration scaffold may have an adhesion strength of 0.2 to 0.5 N/cm², and preferably 0.3 to 0.4 N/cm².

The single or both sides of the cartilage regeneration scaffold may be applied with one or more factors selected from the group consisting of collagens, growth factors, stem cells, exosomes and therapeutic drugs. The above factors applied to the scaffold may improve a regeneration rate of cartilage by promoting cell growth.

Fibrin may be applied to an edge of the cartilage regeneration scaffold. The fibrin applied to the edge may form a fibrous blood clot to serve as a bridge through which cells can move. Through this, cell migration and proliferation in the damaged sites of cartilage may be facilitated to improve the regeneration rate of cartilage.

The cartilage regeneration scaffold may be manufactured by a method including the steps of: applying a polymer solution containing a biocompatible polymer and an organic solvent to a polyurethane acrylate (PUA) mold to prepare a sheet-like semi-cured product; applying pressure to the semi-cured product with a polydimethylsiloxane (PDMS) mold to prepare a patterned semi-cured product; and drying the patterned semi-cured product.

The cartilage regeneration scaffold may be manufactured by a method including the steps of: applying a polymer solution containing a biocompatible polymer and an organic solvent to a polyurethane acrylate (PUA) mold to prepare a sheet-like semi-cured product; placing the semi-cured product between the polyurethane acrylate (PUA) mold and a polydimethylsiloxane (PDMS) mold, and then applying pressure thereto with upper and lower plates to prepare a patterned semi-cured product; and drying the patterned semi-cured product.

The polyurethane acrylate (PUA) mold and/or the polydimethylsiloxane (PDMS) mold have/has concavo-convex portions formed thereon, for forming a pattern on the single side and/or both sides of the scaffold.

More specifically, the cartilage regeneration scaffold may be manufactured by a method including: a semi-cured product forming step (S100), a semi-cured product fixing step (S200), a transfer step (S300), a pressing step (S400), a drying step (S500) and a separation step (S600).

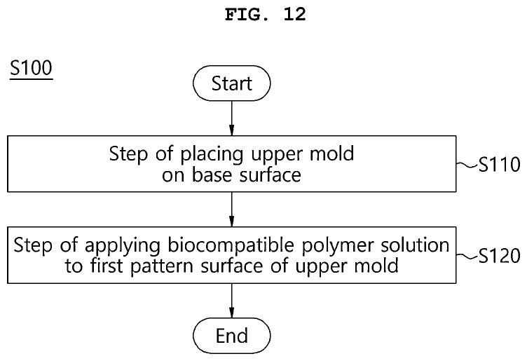

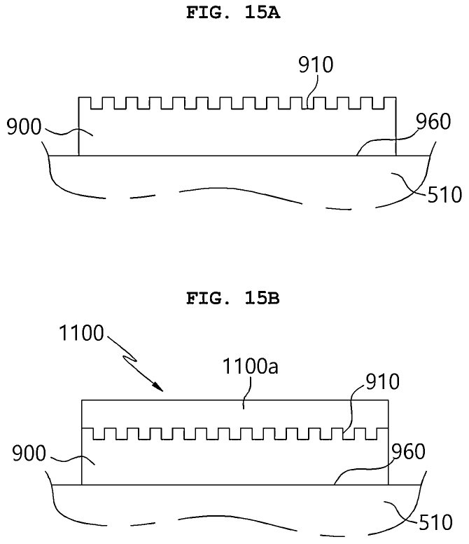

Referring to FIGS. 12 and 15A, the semi-cured product forming step (S100) may include a step (S110) of placing an upper mold 900 on a base surface. The base may be the ground, a structure having a flat surface, or a lower plate 510. When the base surface is a surface of the lower plate 510, the lower plate 510 may be placed on a loading area LA. In this embodiment, the case where the base is the lower plate 510 will be illustrated and described.

The upper mold 900 is a polyurethane acrylate (PUA) mold.

The upper mold 900 may include a first pattern surface 910 on which a pattern is formed, and a first support surface 960 disposed on a surface opposite to the first pattern surface 910. The first support surface 960 may be seated on the base surface.

The first pattern surface 910 may include a pattern of a predetermined shape. The pattern is not limited to a specific shape as long as it has repeating crests and troughs. The pattern may have, for example, a straight line or curved line shape in which crests and troughs are repeated side by side. The pattern may be formed in a shape such as a straight line parallel to any one edge of the cartilage regeneration scaffold, an oblique line forming a predetermined angle with any one edge, a wavy curve and the like.

The width between the crests may be designed variously depending on the pattern of the cartilage regeneration scaffold to be manufactured, such as 600 to 1000 nm, 700 to 1000 nm, 800 to 1000 nm, 600 to 900 nm, 600 to 800 nm, 600 to 700 nm and the like.

The width between the troughs may also be designed to be the same as the width between the crests.

The height difference between the crest and the trough (height of the crest based on the trough) may be designed variously depending on the pattern of the cartilage regeneration scaffold to be manufactured, such as 600 to 1000 nm, 700 to 1000 nm, 800 to 1000 nm, 600 to 900 nm, 600 to 800 nm, 600 to 700 nm and the like.

The distance between the crests, the distance between the troughs, and the height difference between the crest and the trough may be the same as or different from each other. If these are different from each other, differences therebetween may be 100 nm or less, 90 nm or less, 80 nm or less, 70 nm or less, 60 nm or less, or 50 nm or less, respectively.

For example, the distance between the crests, the distance between the troughs, and the height difference between the crest and the trough may be the same as each other such as 800 nm, 700 nm or 600 nm, respectively. In addition, the distance between the crests, and the distance between the troughs may be 800 nm, and the height difference between the crest and the trough may be 750 nm or 850 nm. Further, the distance between the crests and the distance between the troughs may be 700 nm, and the height difference between the crest and the trough may be 650 nm or 750 nm.

The size and spacing of the pattern of the cartilage regeneration scaffold to be manufactured may be changed by adjusting the size and spacing of the pattern formed on the mold.

Referring to FIGS. 12 and 15B, the step (S100) of forming a semi-cured product 1000b may include a step (S120) of applying a biocompatible polymer solution 1000a to the first pattern surface 910 of the upper mold 900. By applying the biocompatible polymer solution 1000a to a PUA mold (the upper mold 900), the sheet-like semi-cured product 1000b may be formed.

The biocompatible polymer solution may be prepared by mixing a biocompatible polymer with an organic solvent. For example, as the organic solvent, halomethane solvents such as chloroform or dichloroform may be used.

An appropriate concentration of the biocompatible polymer solution may vary depending on the type of the biocompatible polymer. For example, the concentration of the biocompatible polymer in the biocompatible polymer solution may be 15% (w/w) to 24% (w/w). When the biocompatible polymer is PLGA, the concentration may be 15% (w/w) to 21% (w/w), and when the biocompatible polymer is PCL, the concentration may be 18% (w/w) to 24% (w/w).

In the process of applying the biocompatible polymer solution 1000a, a part of the solvent evaporates at room temperature to form the sheet-like semi-cured product 1000b, thereby preventing the semi-cured product from flowing down even when turning over the upper mold 900.

As described above, the upper mold 900 in which the semi-cured product 1000b is disposed on the first pattern surface 910 is referred to as a first mold 1100.

Since the semi-cured product 1000b is disposed on the upper mold 900 of the first mold 1100, the first mold 1100 should be turned over later and brought into contact with a lower mold 800.

Therefore, in order to turn over the first mold 1100 and bring the exposed surface of the semi-cured product 1000b into contact with the lower mold 800, a biocompatible polymer with high viscosity is used or a highly volatile solvent is used to allow the solvent quickly to evaporate. Thereby, the viscosity of the semi-cured product 1000b may be increased to prevent it from flowing down.

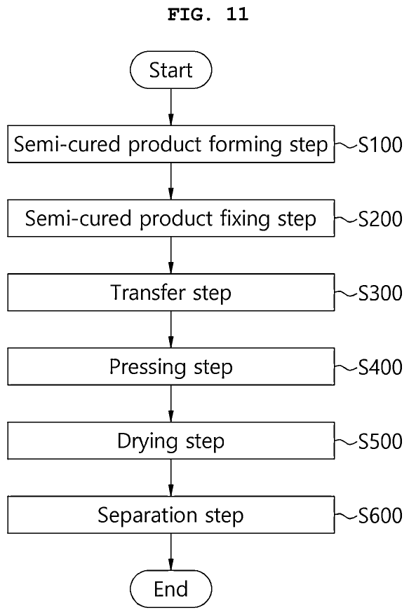

Referring to FIG. 11, the cartilage regeneration scaffold manufacturing method of the present invention may include the step (S200) of fixing the semi-cured product 1000b. Fixing the semi-cured product 1000b is a step of turning over the first mold 1100 and bringing the exposed semi-cured product 1000b into contact with the lower mold 800. The mold formed by bringing the lower mold 800 into contact with the first mold 1100 is referred to as a second mold 1200.

The lower mold 800 is a polydimethylsiloxane (PDMS) mold.

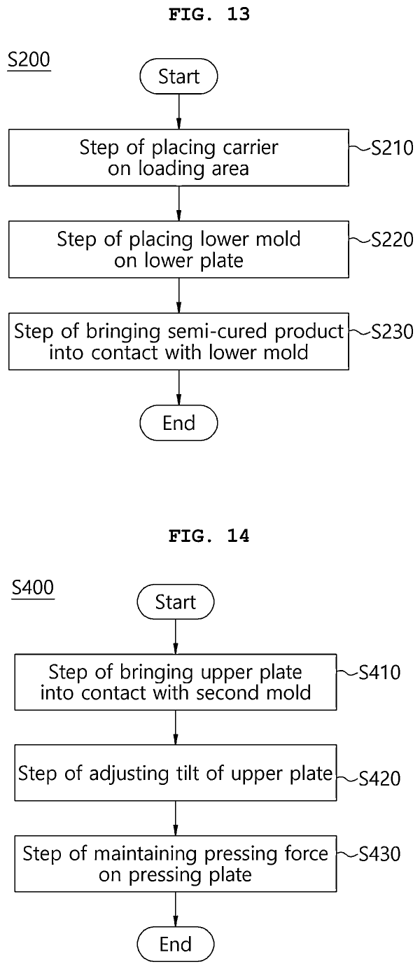

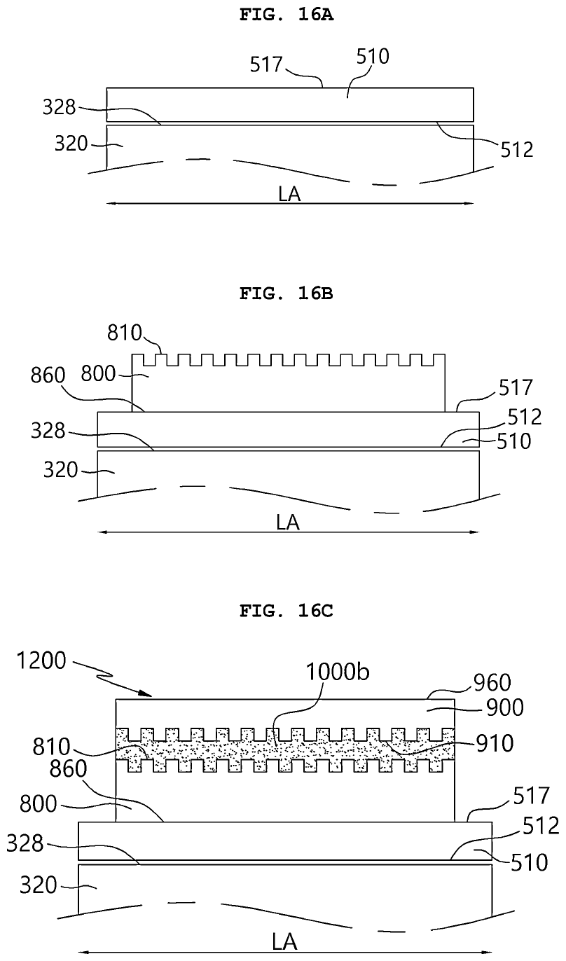

Referring to FIGS. 13 and 16A, the step (S200) of fixing the semi-cured product 1000b may include a step (S210) (of placing a carrier 320 in the loading area LA.

The lower plate 510 may be placed on the carrier 320. For example, a lower plate lower surface 512 may be seated on a carrier seating surface 328. Accordingly, in the loading area LA, the lower plate 510 may be placed so that a lower plate upper surface 517 is exposed to an outside.

Referring to FIGS. 13 and 16B, the step (S200) of fixing the semi-cured product 1000b may include a step (S220) of placing the lower mold 800 on the lower plate 510. For example, in the loading area LA, the lower mold 800 may be placed on the lower plate upper surface 517.

The lower mold 800 may include a flat surface without a pattern and a second support surface 860 formed on a surface opposite to the flat surface. The second support surface 860 may be seated on the lower plate upper surface 517. Accordingly, when the lower mold 800 is placed on the lower plate 510 in the loading area LA, the flat surface may be exposed to the outside. The lower mold 800 having a flat surface without a pattern is used to manufacture a cartilage regeneration scaffold having a nanopattern formed on a single side (“single-sided nanopatterned cartilage regeneration scaffold”).

The lower mold 800 may include a second pattern surface 810 and a second support surface 860 formed on a surface opposite to the second pattern surface 810. The second pattern surface 810 may be a flat surface without a pattern or a surface on which a pattern is formed. In the case of the flat surface without a pattern, it is used when manufacturing a single-sided patterned cartilage regeneration scaffold, and in the case of the surface on which the pattern is formed, it is used when manufacturing a double-sided patterned cartilage regeneration scaffold. The second support surface 860 may be seated on the lower plate upper surface 517. Accordingly, when the lower mold 800 is placed on the lower plate 510 in the loading area LA, the second pattern surface 810 may be exposed to the outside.

Referring to FIGS. 13 and 16C, the step (S200) of fixing the semi-cured product 1000b may include a step (S230) of bringing the first mold 1100 into contact with the lower mold 800. For example, the first mold 1100 may be placed so that the semi-cured product 1000b brings into contact with the second pattern surface 810, thus fixing the semi-cured product.

In the step (S200) of fixing the semi-cured product 1000b, the first support surface 960 of the upper mold 900 may be exposed to the outside in the loading area LA.

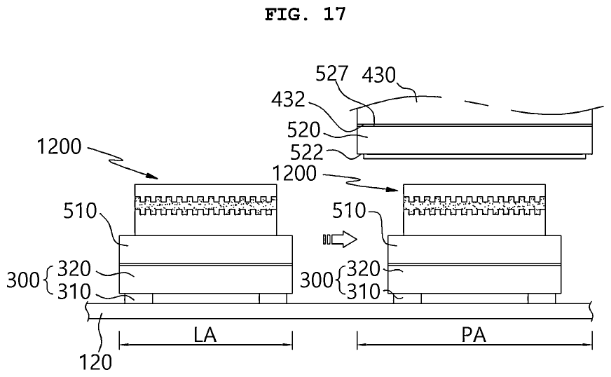

Referring to FIGS. 11 and 17, the cartilage regeneration scaffold manufacturing method according to an embodiment of the present invention may include the transfer step (S300) of transferring the second mold 1200.

In the transfer step (S300), the second mold 1200 placed in the loading area LA may be moved to a molding area PA.

A pressing plate 430 of a pressing unit 400 may be disposed in an upper section of the molding area PA. The pressing plate 430 may be coupled with an upper plate 520. Accordingly, the second mold 1200 may be disposed below the upper plate 520.

For example, the pressing plate 430 may include a pressing plate lower surface 432 and a pressing plate upper surface 437. The pressing plate lower surface 432 may be coupled with the upper plate 520.

The upper plate 520 may include an upper plate pressing surface 522 and an upper plate coupling surface 527. The upper plate coupling surface 527 may be coupled with the pressing plate lower surface 432. In addition, a pressure dispersion part 550 may be adhered to the upper plate pressing surface 522.

A transfer unit 300 which transfers the second mold 1200 by moving the second mold 1200, and the lower plate 510 disposed on the transfer unit 300 may be disposed in a lower section of the molding area PA.

For example, the transfer unit 300 may include a transfer rail 310 and a carrier 320. The lower plate 510 may be disposed on the carrier 320. The second mold 1200 may be disposed on the lower plate 510. Accordingly, the second mold 1200 may be disposed in the molding area PA.

Further, the transfer unit 300 may further include a transfer drive part 330 capable of moving the carrier 320. The carrier 320 may be moved from the loading area LA to the molding area PA by driving the transfer drive part 330.

Accordingly, in the molding area PA, the second mold 1200 may be disposed in a downward direction of the pressure dispersion part 550. For example, in the transfer step (S300), the pressure dispersion part 550 and the first support surface 960, which is an exposed surface of the second mold 1200, may be disposed to face each other in the molding area PA.

Referring to FIG. 11, the cartilage regeneration scaffold manufacturing method according to an embodiment of the present invention may include the pressing step (S400) of pressing the fixed semi-cured product 1000b.

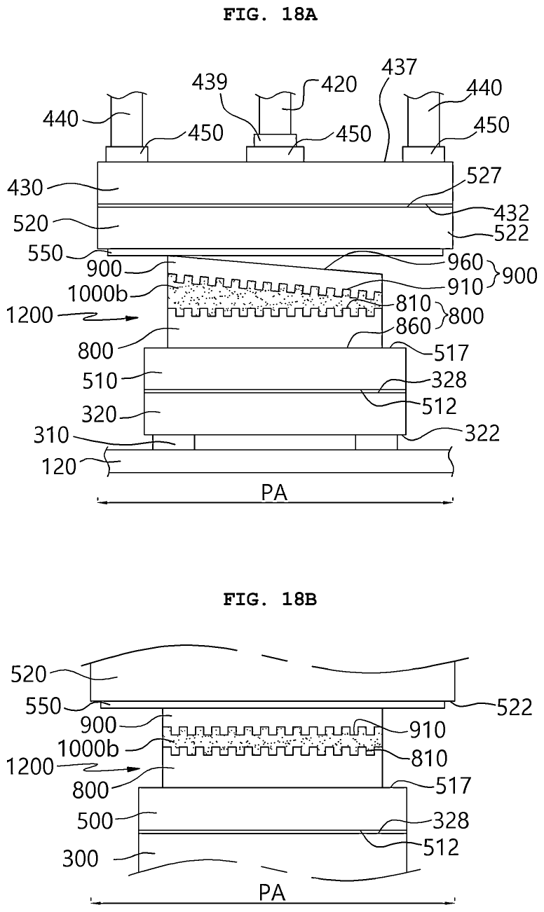

For example, referring to FIGS. 14 and 18A, the pressing step (S400) may be performed in the molding area PA.

The pressing step (S400) of pressing the fixed semi-cured product 1000b may include a step (S410) of bringing the upper plate 520 into contact with the second mold 1200. For example, the upper plate pressing surface 522 of the upper plate 520 may be brought into contact with the first support surface 960 of the upper mold 900. For a specific example, the step (S410) of contacting the upper plate 520 may bring the pressure dispersion part 550 into contact with the first support surface 960.

The step (S410) of bringing the upper plate 520 into contact with the second mold 1200 may include a step of operating the pressing unit 400. For example, the pressing unit 400 may operate an actuator installed in the pressing unit body 410. A pressing rod 420 may be connected to the actuator. The pressing rod 420 may be moved toward the pressing plate 430. The pressing rod 420 may be seated on a seating part 439 disposed on one surface of the pressing plate 430.

The pressing rod 420 may move in a vertical direction. When the pressing rod 420 moves in the downward direction, the upper plate 520 coupled to the pressing plate 430 may move in the downward direction. Accordingly, the upper plate 520 may be moved toward the lower plate 510.

The upper plate 520 may be moved to bring the upper plate pressing surface 522 into contact with the second mold 1200 disposed on the lower plate 510. More specifically, the pressure dispersion part 550 may be brought into contact with the first support surface 960 of the upper mold 900 exposed in the second mold 1200.

In the step (S410) of bringing the upper plate 520 into contact with the second mold 1200, the case where some areas of the second mold 1200 have different thicknesses from other areas may occur.

For example, during the application process of the biocompatible polymer solution 1000a, when the biocompatible polymer solution 1000a is unevenly dispersed, a thickness difference may occur. Alternatively, the thickness difference may occur between the formed thicknesses of the upper mold 900 and the lower mold 800.

When the above thickness difference occurs, the upper plate 520 may be tilted, such that the whole surface of the pressure dispersion part 550 and the whole surface of the first support surface 960 may not be brought into surface contact. Referring to FIGS. 14 and 18B, the pressing step (S400) may include a step (S420) of adjusting a tilt of the upper plate 520.

In the step (S420) of adjusting the tilt, the upper plate 520 and the fixed semi-cured product 1000b may be brought into surface contact. For example, in the step (S420) of adjusting the tilt of the upper plate 520, the whole surface of the pressure dispersion part 550 and the whole surface of the first support surface 960 may be brought into surface contact.

After bringing the two components into surface contact, a step of leveling the upper plate 520 may be performed. That is, rotation (tilt adjustment) of the upper plate 520 may be performed.

The tilt of the upper plate 520 may be adjusted through a gimbal part 450. The gimbal part 450 may be disposed on an upper surface of the pressing plate 430. For example, the gimbal part 450 may be disposed between guide rods 440 and the pressing plate 430 formed in the pressing unit 400. In addition, the gimbal part 450 may be disposed between the seating part 439 and the pressing plate 430.

If the tilt of the upper plate 520 is not leveled, the gimbal part 450 may rotate the upper plate 520 to level the upper plate 520.

Accordingly, the gimbal part 450 may provide a load of the upper plate 520 to an area of the second mold 1200 where the thickness difference occurs. For example, the gimbal part 450 may rotate (tilt) the upper plate 520 to provide a load to the area where the thickness difference occurs. Here, since the upper plate 520 is coupled to the pressing plate 430, the loads of the upper plate 520 and the pressing plate 430 may be provided to the second mold 1200.

The loads of the upper plate 520 and the pressing plate 430 provided to the second mold 1200 may apply pressure to the fixed semi-cured product 1000b. Accordingly, the loads of the upper plate 520 and the pressing plate 430 may press the fixed semi-cured product 1000b in the area where the thickness difference occurs.

Once the upper plate 520 is leveled, the gimbal part 450 may stop providing a load to the second mold 1200.

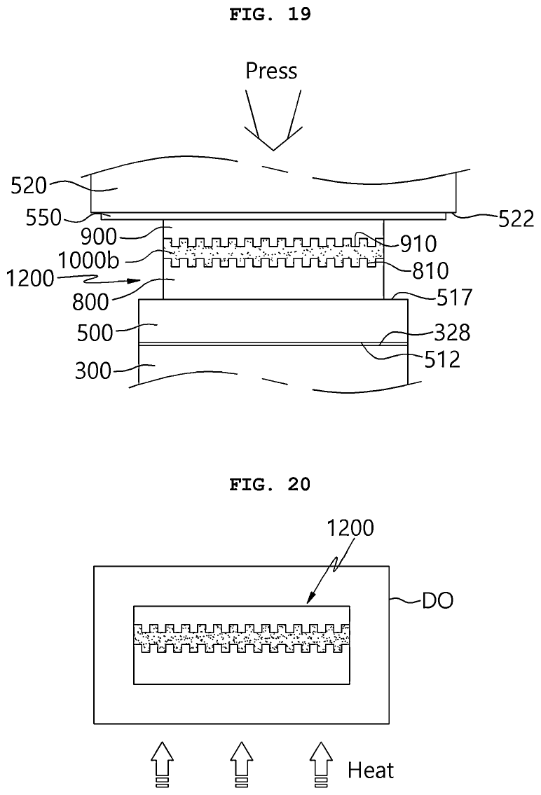

Referring to FIGS. 14 and 19, the inventive method may include a step (S430) of maintaining the pressing force on the pressing plate 430.

The step (S430) of maintaining the pressing force may be performed in a state where the pressure dispersion part 550 and the first support surface 960 are in surface contact and the upper plate 520 is leveled.

In the above-described state, the target pressing force may be maintained on the pressing plate 430. In the step of maintaining the pressing force (S430), a pressing force of 0.40 to 0.70 Pa, preferably 0.45 to 0.65 MPa, may be provided to the pressing plate 430. The time for providing the pressing force may vary depending on the magnitude of the pressing force, and for example, the pressing force may be provided for a maintaining time of 30 to 50 minutes.

The target pressing force provided to the pressing plate 430 may be transmitted to the upper plate 520. The pressing force transmitted to the upper plate 520 may be provided to the second mold 1200 through the upper plate pressing surface 522. The pressing force provided to the second mold 1200 may be transmitted to the fixed semi-cured product 1000b.

The pattern shapes formed on the first pattern surface 910 and the second pattern surface 810 may be transferred to the semi-cured product 1000b through the pressing force provided to the second mold 1200.

Referring to FIGS. 11 and 20, the inventive method may include the drying step (S500) of drying the semi-cured product 1000b.

In the drying step (S500), target drying heat may be provided to the second mold 1200 through the lower plate 510. Alternatively, the second mold 1200 may be dried in a dry oven or a hot plate, and consequently drying the semi-cured product 1000b.

When drying the second mold 1200 through the lower plate 510, the lower plate 510 may be provided with a heating plate therein. The heating plate may transmit heat energy to the lower plate upper surface 517 to dry the second mold 1200. The heat energy transmitted to the second mold 1200 may be applied to the semi-cured product 1000b to dry the semi-cured product 1000b.

Meanwhile, when drying the second mold 1200 in the dry oven, the carrier 320 may be moved from the molding area PA to the loading area LA, and the second mold 1200 loaded on the lower plate 510 may be unloaded in the loading area LA. The second mold 1200 may be unloaded and then placed in a dry oven DO, and the drying heat may be provided to the second mold 1200.

Here, the drying step (S500) may provide heat energy of 15° C. to 45° C. to the second mold 1200 for 6 to 10 hours.

On the other hand, in the drying step (S500), the second mold 1200 may be placed on a hot plate to be dried. When using the hot plate, a 0.5 to 2 Kg steel plate is stacked on the second mold 1200, and the hot plate may be heated to 60° C. to 80° C., thus drying the second mold 1200. The second mold 1200 may be dried while maintaining it at the above heating temperature for 4 to 8 hours.

Here, the steel plate may prevent the upper mold 900 or the lower mold 800 from being separated from the semi-cured product 1000b during the drying process.

If the upper mold 900 or the lower mold 800 is separated during the drying process, the pattern on the surface of the semi-cured product 1000b may be damaged. Therefore, the drying process may be performed by placing the steel plate on the second mold 1200 to prevent the pattern on the surface of the semi-cured product 1000b from being damaged.

As such, the second mold 1200 may be dried to form a cured cartilage regeneration scaffold 1000 between the upper mold 900 and the lower mold 800.

Referring to FIGS. 11 and 20, the step (S600) of separating the cartilage regeneration scaffold 1000 from the second mold 1200 may be performed.

The cartilage regeneration scaffold 1000 formed between the lower mold 800 and the upper mold 900 constituting the second mold 1200 may be separated from the lower mold 800 and the upper mold 900.

After the separation step (S600), the cartilage regeneration scaffold 1000 may be immersed in 70% ethanol for 10 seconds and washed with purified water.

The cartilage regeneration scaffold manufacturing method of the present invention may be performed using a cartilage regeneration scaffold manufacturing apparatus 10 as follows. Hereinafter, the cartilage regeneration scaffold manufacturing apparatus will be described in detail with reference to the drawings.

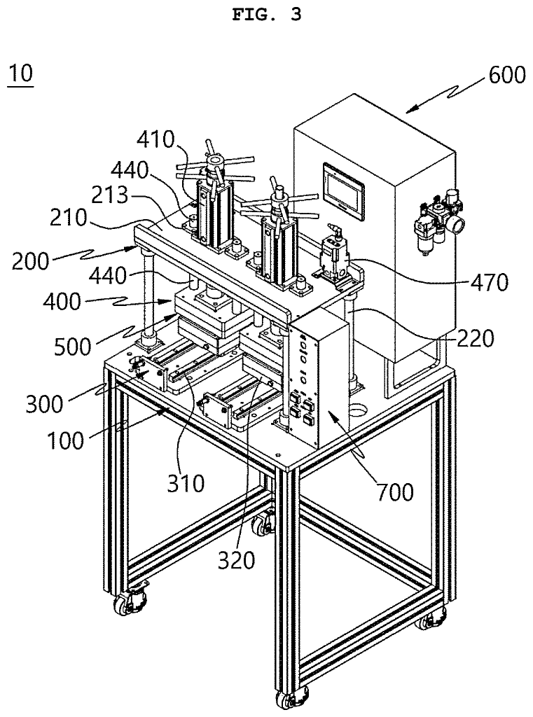

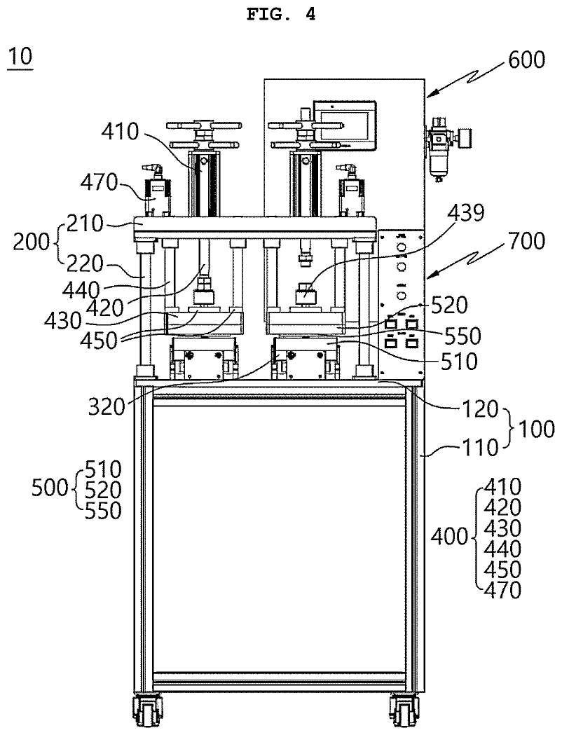

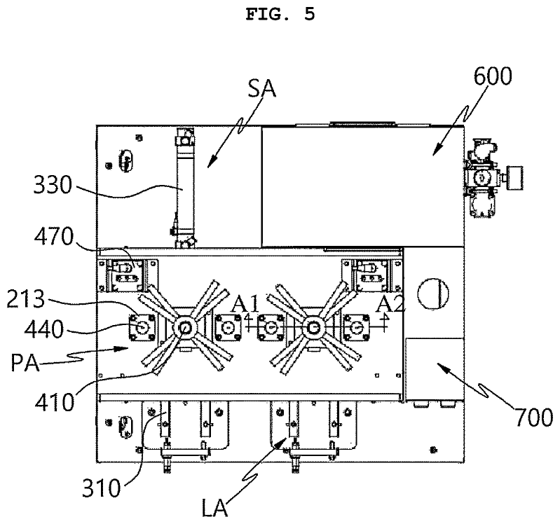

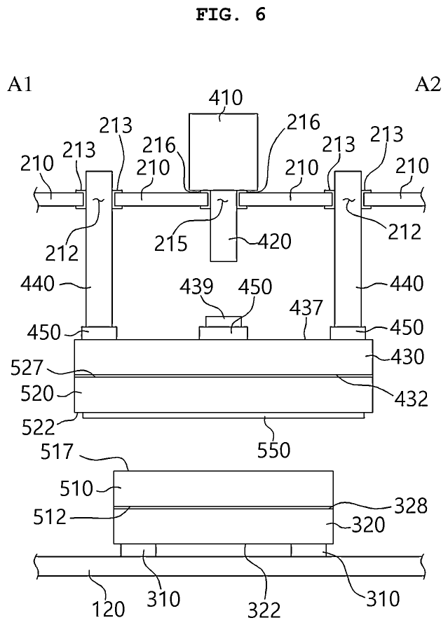

FIG. 3 is a perspective view illustrating the cartilage regeneration scaffold manufacturing apparatus according to an embodiment of the present invention, FIG. 4 is a front view illustrating the cartilage regeneration scaffold manufacturing apparatus according to an embodiment of the present invention, FIG. 5 is a top view illustrating the cartilage regeneration scaffold manufacturing apparatus according to an embodiment of the present invention, and FIG. 6 is a cross-sectional view taken on A1-A2 in FIG. 5.

Referring to FIGS. 3 to 6, the cartilage regeneration scaffold manufacturing apparatus 10 may include a stand 100.

The stand 100 may include a stand body 110 and a table 120.

The table 120 may be formed as a plate having a flat surface. A plurality of components may be placed on the table 120. The flat surface may provide a stable seating surface for the components.

The stand body 110 may be provided with a plurality of legs. The plurality of legs may be disposed at edge regions of the table 120. The plurality of legs may be disposed at different set lengths or at the same set length. The plurality of legs with adjusted set lengths may form a flat surface of the table 120.

In addition, the plurality of legs may be disposed at a set length corresponding to working heights of workers. For example, the plurality of legs may be disposed at the expected working heights of the workers. The plurality of legs with adjusted heights may improve the workability of the workers.

The cartilage regeneration scaffold manufacturing apparatus 10 may include a frame unit 200. The frame unit 200 may be placed on the table 120. The frame unit 200 may include a frame support 220 and a frame body 210.

The frame support 220 may support the frame body 210. The frame support 220 may be disposed on an upper surface of the table 120. The frame support 220 is installed on the table 120 to fix the frame body 210. The frame support 220 and the table 120 may be fixed to each other through a fastening structure such as a fixing screw, but the fixing means is not limited to the fixing screw. In addition, the frame support 220 and the frame body 210 may also be fixed to each other using similar fixing means.

The frame body 210 may be disposed on the table 120. For example, the frame body 210 is located on the upper surface of the table 120 and may be supported by the frame support 220. The frame body 210 may be formed and disposed in a plate shape. Accordingly, the frame body 210 may be disposed in a direction parallel to the flat surface of the table 120.

The frame body 210 may have a plurality of through holes 212 and 215. The frame body 210 may include cylinder guides 213 and 216 installed into the plurality of through holes 212 and 215, respectively.

The plurality of through holes 212 and 215 may include a first through hole 212 and a second through hole 215. The guide rod 440 may be installed in the first through hole 212. The pressing rod 420 may be installed in the second through hole 215.

A plurality of first through holes 212 may be arranged around the second through hole 215. The first through holes 212 may be symmetrically arranged around the second through hole 215.

In this embodiment, the case where two first through holes 212 are symmetrically arranged around the second through hole 215 will be illustrated and described. However, the arrangement of the first through holes 212 is not limited thereto, and three to eight first through holes 212 may be symmetrically arranged around the second through hole 215.

The guide rod 440 may disperse the concentrated pressure provided from the pressing rod 420 toward the surface of the pressing plate 430. Therefore, since the number of arranged first through holes 212 is related to the number of arranged guide rods 440, it may serve to form pressure dispersed on the pressing plate 430. The pressing plate 430 with dispersed pressure may help to form a uniform surface pressure in a molding unit 500.

The cartilage regeneration scaffold manufacturing apparatus 10 may include the transfer unit 300.

Here, for the convenience of description, in the table 120, an area housed and surrounded by the frame support 220 and the frame body 210 is defined as the molding area PA, and areas other than the molding area PA is defined as surrounding areas SA. Among the surrounding areas SA, an area where the transfer unit 300 is disposed is defined as the loading area LA.

The transfer unit 300 may include the transfer rail 310, the carrier 320 and the transfer drive part 330.

The transfer rail 310 may be disposed on the upper surface of the table 120. The transfer rail 310 may be disposed from the loading area LA to the molding area PA. The transfer rail 310 may form a rail part with a pair of rails disposed in a direction from the loading area LA to the molding area PA. At least one or more rail parts may be disposed. For example, when the transfer rail 310 has a plurality of rail parts formed in the direction from the loading area LA to the molding area PA, the loading area LA may form a plurality of work ports.

The carrier 320 may be mounted on the transfer rail 310. The carrier 320 may move along the transfer rail 310. Accordingly, the carrier 320 may be placed in the loading area LA and the molding area PA.

The carrier 320 may be movably mounted on the transfer rail 310. For example, the carrier 320 may be slidably mounted on the transfer rail 310, but it is not limited thereto, and the carrier 320 may be mounted on the transfer rail 310 using any structure as long as it is a movable means.

The carrier 320 may include a sliding part 322 having a sliding structure, and the seating surface 328 formed on a surface opposite to the sliding part 322. The sliding part 322 may be mounted on the transfer rail 310. The lower plate 510 may be seated on the seating surface 328. The seating surface 328 may face the lower plate 510.

The carrier 320 may be connected to the transfer drive part 330. The transfer drive part 330 may be connected to a thickness surface formed between the sliding part 322 and the seating surface 328.

The transfer drive part 330 may be arranged in the surrounding area SA. The transfer drive part 330 may be equipped with an actuator to move the carrier 320 to the loading area LA and molding area PA. In this embodiment, a structure in which the actuator moves forward and backward is illustrated and described, but in some cases, the carrier 320 may be moved through a structure in which the actuator moves left and right.

The cartilage regeneration scaffold manufacturing apparatus 10 may include the pressing unit 400. The pressing unit 400 may be disposed in the molding area PA. The pressing unit 400 may include the pressing unit body 410, the pressing rod 420, the pressing plate 430 and the guide rod 440.

The pressing unit body 410 may be disposed on an upper surface of the frame body 210. The pressing unit body 410 may include an actuator. The actuator may move the pressing rod 420 in the vertical direction.

The pressing rod 420 may be installed in the second through hole 215 that penetrates upper and lower surfaces of the frame body 210. For example, the pressing rod 420 may be inserted into the second cylinder guide 216 installed in the second through hole 215.

The pressing rod 420 may move in the vertical direction. The pressing rod 420 may move to bring into contact with the pressing plate 430. The pressing rod 420 may move the pressing plate 430 downward. The pressing rod 420 may apply pressure to the pressing plate 430 when it encounters an object to be molded in the target area.

The pressing unit 400 may include the pressing plate 430 disposed between the frame body 210 and the table 120.

The pressing plate 430 may include the pressing plate upper surface 437 with which the pressing rod 420 brings into contact. The pressing plate upper surface 437 may be provided with the seating part 439. The pressing rod 420 may be seated on the seating part 439.

The seating part 439 may be disposed in an area including a center of gravity of the pressing plate 430. For example, when the pressing plate 430 is disposed in a square shape, the center of gravity of the pressing plate 430 may be a central area of the square shape. Accordingly, the seating part 439 may be disposed in the central area.

One end of the pressing rod 420 may be seated on the seating part 439. The seating part 439 may be formed on the pressing plate upper surface 437 and made of a material capable of withstanding the pressing force, such as rubber or silicone.

As such, the pressing rod 420 moves in the downward direction and the pressing rod 420 may move the pressing plate 430 downward. In addition, when the pressing plate 430 moves to the target location, the pressing rod 420 may apply a pressing force to the center of gravity of the pressing plate 430.

The guide rods 440 may be arranged around the pressing rod 420. The guide rod 440 may be disposed between the frame body 210 and the pressing plate 430.

One end of the guide rod 440 may be coupled to the pressing plate 430. The other end of the guide rod 440 may be inserted into the first through hole 212 penetrating a portion of the frame body 210. For example, the first cylinder guide 213 may be installed in the first through hole 212 formed in the frame body 210. The guide rod 440 may be inserted into the first cylinder guide 213.

As described above, the plurality of first through holes 212 may be symmetrically arranged around the second through hole 215. The guide rods 440 may be installed in each of the plurality of first through holes 212.

Accordingly, the plurality of guide rods 440 may be symmetrically arranged about the pressing rod 420. The plurality of guide rods 440 may serve to disperse the pressing force so as to form a uniform surface pressure by the molding unit 500. The plurality of guide rods 440 may form a uniform pressing force on the whole surface of the pressing plate 430.

Meanwhile, the gimbal part 450 may be disposed between the guide rod 440 and the pressing plate 430. For example, the gimbal part 450 may connect the lower surface of the guide rod 440 and the upper surface of the pressing plate 430. In addition, the gimbal part 450 may be selectively disposed between the seating part 439 and the pressing plate 430. The gimbal part 450 may connect the lower surface of the seating part 439 and the upper surface of the pressing plate 430.

The gimbal parts 450 may be symmetrically arranged around the seating part 439 centered the seating part 439. In other words, as the plurality of guide rods 440 are arranged around the area including the center of gravity of the pressing plate 430 on the upper surface of the pressing plate 430, the gimbal parts 450 disposed on each of the plurality of guide rods 440 may also be arranged in the same arrangement structure.

The gimbal part 450 may adjust the tilt of the pressing plate 430. For example, the gimbal part 450 may level the upper plate 520. That is, the gimbal part 450 may rotate (adjust the tilt of) the upper plate 520.

If the tilt of the upper plate 520 is not leveled, the gimbal part 450 may rotate the upper plate 520 to level the upper plate 520.

Accordingly, the gimbal part 450 rotates (adjusts the tilt of) the upper plate 520 to level it, thereby providing the load of the upper plate 520 to the second mold (see 1200 in FIGS. 18A and 18B). For example, since the upper plate 520 is coupled to the pressing plate 430, the loads of the upper plate 520 and the pressing plate 430 may be applied to the second mold 1200.

The gimbal part 450 may adjust pressure imbalance that may occur due to a thickness difference of the object to be molded. For example, when the thicknesses of areas in the object to be molded are different, the gimbal part 450 may adjust the tilt of the pressing plate 430 so that the pressing plate 430 corresponds to the whole surface of one surface of the object to be molded.

Accordingly, when the thickness of some areas and the thickness of other areas of the object to be molded are different, the gimbal part 450 may adjust the pressing plate 430 to prevent the pressing force from being concentrated in some areas of the object to be molded or more pressing force than required from being transmitted thereto.

As such, the gimbal part 450 may adjust the tilt of the pressing plate 430 to improve the molding uniformity of the object to be molded.

The cartilage regeneration scaffold manufacturing apparatus 10 may include the molding unit 500. The molding unit 500 may include the lower plate 510, the upper plate 520 and the pressure dispersion part 550.

The lower plate 510 may be disposed on the upper surface of the carrier 320. For example, the lower plate 510 may be seated on the seating surface 328 of the carrier 320. The lower plate 510 may be formed in a shape similar to or smaller than that of the carrier 320.

The lower surface 512 of the lower plate 510 may be seated on the seating surface 328 of the carrier 320. The carrier 320 may move between the loading area LA and the molding area PA along the transfer rail 310. Accordingly, the lower plate 510 seated on the carrier 320 may move between the loading area LA and the molding area PA through the carrier 320.

The lower plate 510 may include the upper surface 517 of the lower plate 510 on a surface opposite to the lower surface 512 of the lower plate 510. The lower mold (see 800 in FIGS. 18A and 18B) may be disposed on the upper surface 517 of the lower plate 510. The manufacturing method regarding the lower mold 800 will be described in detail.

The upper plate 520 may be disposed in the molding area PA. The upper plate 520 may be coupled to the pressing plate 430. The upper plate 520 may include the upper plate coupling surface 527 to be coupled to the pressing plate 430. For example, the upper plate coupling surface 527 may be coupled to the lower surface 432 of the pressing plate 430. The pressing plate lower surface 432 may be a surface facing the pressing plate upper surface 437 on which the seating part 439 is disposed.

The shape of the upper plate 520 may be formed to have a larger area than the shape of the lower plate 510. The object to be molded may be placed between the upper plate 520 and the lower plate 510. Since the upper plate 520 has a larger area than the shape of the lower plate 510, it may provide uniform pressure to the object to be molded. Therefore, the size of the object to be molded may be limited by the shape of the lower plate 510.

As described above, the cartilage regeneration scaffold manufacturing apparatus 10 according to the present invention may disperse the pressing force to implement the pressing plate 430 on which a uniform pressing force is formed, thereby increasing the area of the lower plate 510. Therefore, the object to be molded which is placed on the lower plate 510 may be formed in a large area.

The upper plate 520 may include an upper plate pressing surface 522 on a surface opposite to the upper plate coupling surface 527. The pressure dispersion part 550 may be disposed on the upper plate pressing surface 522.

The pressure dispersion part 550 may be adhered to the upper plate pressing surface 522 through an adhesive. The pressure dispersion part 550 may be made of a silicone material, but it is not limited thereto, and any material may be used as long as it can transmit the pressure. The pressure dispersion part 550 may evenly disperse the pressing force applied from the pressing plate 430 on the whole surface of the upper plate 520.

The cartilage regeneration scaffold manufacturing apparatus 10 may include an input unit 600 and a display unit 700. The input unit 600 and display unit 700 may be arranged in the surrounding area SA.

The cartilage regeneration scaffold manufacturing apparatus 10 may include the input unit 600 configured to receive input from a worker. For example, the worker may directly input set values into the input unit 600.

The input unit 600 may include input means such as an input button for inputting the set values. Here, the set value may include, for example, the pressure of the pressing unit 400, the heating temperature of the lower plate 510, the pressure provision time and heating time of the pressing unit 400, etc.

The display unit 700 may display the set value on a screen. In other words, the display unit 700 may display input information on the screen. For example, the display unit 700 may display the pressure of the pressing unit 400, the heating temperature of the lower plate 510, the pressure provision time and the heating time, etc., in numerical values.

In addition, a controller capable of controlling the set values of the cartilage regeneration scaffold manufacturing apparatus 10 may be further arranged in the space where the display unit 700 is disposed.

As such, the cartilage regeneration scaffold manufacturing apparatus 10 according to an embodiment of the present invention may form a uniform pressure, thus manufacturing a cartilage regeneration scaffold with improved uniformity.

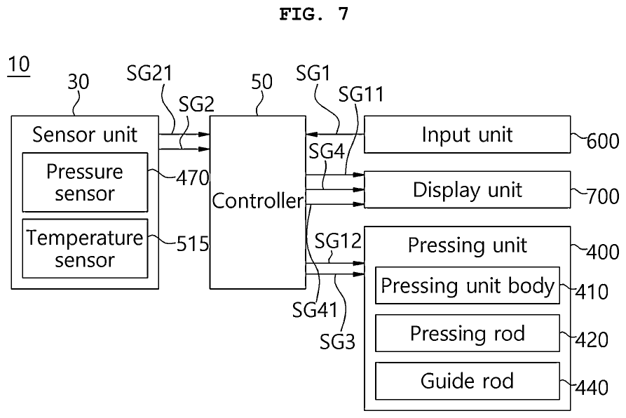

FIG. 7 is a block diagram illustrating the cartilage regeneration scaffold manufacturing apparatus according to an embodiment of the present invention.

FIG. 7 will be described by referring to FIGS. 3 to 6 in order to avoid redundant description and facilitate understanding of the description.

The cartilage regeneration scaffold manufacturing apparatus 10 according to an the present embodiment of invention may include a sensor unit 30 and the controller 50.

The controller 50 may be connected to the display unit 700, the input unit 600 and the sensor unit 30. The controller 50 may be arranged in the area where the display unit 700 is displaced.

The controller 50 may also be connected to a configuration in which sensor unit 30 is disposed. For example, the controller 50 may be connected a configuration in which a pressure sensor 470 and/or a temperature sensor 515 are displaced.

For a specific example, when the temperature sensor 515 is disposed in the lower plate 510, the controller 50 may be connected to the lower plate 510. For another example, when the pressure sensor 470 is disposed in the pressing unit 400, the controller 50 may be connected to the pressing unit 400. The sensor unit 30 may include the pressure sensor 470. The pressure sensor 470 may be disposed on the upper surface of the frame body 210.

In this drawing, the case where the pressure sensor 470 is disposed in the pressing unit 400 will be illustrated and described. Here, the pressure sensor 470 may also be disposed in the molding unit 500.

The input unit 600 may receive input from the worker. The input unit 600 may input the pressure provided to the pressing rod 420 of the pressing unit 400. The provided input may be a set pressure at which the pressing unit 400 operates. The input unit 600 may generate a first signal SG1 consisting of the set pressure.

The input unit 600 may provide the first signal SG1 to the controller 50. The controller 50 may convert the received first signal SG1 and transmit it to the display unit 700 and the pressing unit 400.

The controller 50 may convert the first signal SG1 into a first 1 signal SG11 and transmit it to the display unit 700. The display unit 700 that has received the first-1 signal SG11 may display the set pressure on the screen.

In addition, the controller 50 may convert the first signal SG1 into a first-2 signal SG12 and transmit it to the pressing unit 400.

The pressing unit 400 may operate according to the first-2 signal SG12. For example, the pressing unit 400 may operate the pressing rod 420 according to the first-2 signal SG12. For example, the pressing rod 420 may move the pressing plate 430 according to the first-2 signal SG12. When the pressing plate 430 moves according to the first-2 signal SG12 and brings into contact with the object to be molded, the pressing rod 420 may provide pressure to the pressing plate 430.

The pressure sensor 470 of the sensor unit 30 may sense the pressure formed in the pressing unit 400. The sensor unit 30 may generate a second signal SG2 using information on sensing the pressure formed in the pressing unit 400. The sensor unit 30 may transmit the second signal SG2 to the controller 50.

The controller 50 that has received the second signal SG2 may determine whether the information on the pressure according to the first-2 signal SG12 has been transmitted through the pressing unit 400 through calculation. For example, the controller 50 may calculate a difference between a pressure set in the pressing rod 420 and the pressure received from the pressure sensor 470. The controller 50 may generate a third signal SG3 and a fourth signal SG4 from a value obtained by calculating the pressure difference (hereinafter “calculated value”).

If the calculated value is within an error range by comparing with the previously measured data, the controller 50 may transmit the fourth signal SG4 to the display unit 700. In addition, the controller 50 may generate a third signal SG3 which is the same as the first-2 signal SG12 and provide the third signal SG3 to the pressing unit 400.

Meanwhile, if the calculated value deviates from the error range by comparing with the previously measured data, the controller 50 may provide a corrected input value to the pressing unit 400.

Here, when the controller 50 is manually set, it is possible to transmit the fourth signal SG4 consisting of the calculated value to the display unit 700. The display unit 700 may display the calculated value according to the fourth signal SG4 on the screen.

The worker may check the calculated value displayed on the display unit 700. The worker may input the corrected input value into the input unit 600 based on the displayed information.

The controller 50 may generate a third signal SG3 using the corrected input value. The third signal SG3 generated in the controller 50 may be transmitted to the pressing unit 400. Through the third signal SG3, the pressing unit 400 may provide the corrected pressure value to the pressing plate 430. The corrected pressure value may be the pressure to be provided to the pressing plate 430.

In addition, the controller 50 may generate a fourth-1 signal SG41 based on information according to the third signal SG3. The fourth-1 signal SG41 may be provided to the display unit 700. The display unit 700 may display information consisting of the fourth-1 signal SG41 on the screen.

As such, the cartilage regeneration scaffold manufacturing apparatus 10 according to the present invention may control the pressure provided to the pressing unit 400 through the information from the sensor unit 30.

Meanwhile, when the controller 50 is automatically set, if a value obtained by calculating the pressure difference (hereinafter referred to as “calculated value”) has a difference, the fourth signal SG4 may not be generated.

The pressure sensor 470 may sense the pressure formed in the pressing unit 400. The sensor unit 30 may generate a second-1 signal SG21 through the sensing information. The sensor unit 30 may transmit the second-1 signal SG21 to the controller 50.

The controller 50 may compare the calculated value with the previously measured data to determine whether it is within the error range based on the previously measured data. If it is determined that the calculated value is within the error range, the controller 50 may generate a third signal SG3 which is the same as the first-2 signal SG12.

The third signal SG3 generated in the controller 50 may be transmitted to the pressing unit 400. Through the third signal SG3, the pressing unit 400 may provide pressure according to the first-2 signal SG12 to the pressing plate 430. The pressure according to the first-2 signal SG12 may be the pressure provided to the pressing plate 430.

In addition, the controller 50 may generate a fourth signal SG4 based on information according to the third signal SG3. The fourth signal SG4 may be provided to the display unit 700. The display unit 700 may display information consisting of the fourth signal SG4 on the screen.

As such, the cartilage regeneration scaffold manufacturing apparatus 10 according to the present invention may control the pressure provided to the pressing unit 400 through the information from the sensor unit 30.

On the other hand, the controller 50 may compare the input pressure with the pressure applied to the object to be molded. For example, the pressing unit 400 may provide pressure to the molding unit 500. For example, the pressing unit 400 may transmit the pressure formed in the pressing plate 430 to the molding unit 500.

A difference between the pressure formed in the pressing unit 400 and the pressure formed in the molding unit 500 may occur. The controller 50 may compare the pressure provided from the pressing unit 400 and the pressure output from the molding unit 500. The output pressure may be the pressure applied to the object to be molded.

The provided pressure may be transmitted to the molding unit 500 through the pressing plate 430. The output pressure applied to the object to be molded may be formed through the molding unit 500.

The pressure sensor 470 of the sensor unit 30 may be disposed in each of the pressing unit 400 and the molding unit 500. The pressure sensor 470 may sense the provided pressure of the pressing unit 400. In addition, the pressure sensor 470 may sense the output pressure of the molding unit 500.

The sensor unit 30 may generate a second signal consisting of the provided pressure and the output pressure.

There may be a difference between the provided pressure input from the pressing rod 420 and the output pressure output from the molding unit 500 (hereinafter “pressure difference”). For example, a pressure difference may occur in the process of dispersing pressure to the whole surface of the pressing plate 430 or in the process of dispersing pressure by the pressure dispersion part 550.

In this case, the probability of molding defects occurred in molded products may be increased. In addition, loss caused by leakage of power used in the manufacturing apparatus may occur.

The pressure sensor 470 may sense the pressure in each set area, and the sensor unit 30 may convert the pressure sensed in each set area into a signal as the second signal SG2, and transmit it to the controller 50.

The controller 50 may calculate the pressure difference. The set value of the pressing unit 400 may be readjusted by reflecting the calculated value. In addition, the coupling structure between the components may be readjusted. Therefore, the pressure sensor 470 may reduce the probability of defects and decrease the loss caused by leakage of power used in the manufacturing apparatus.

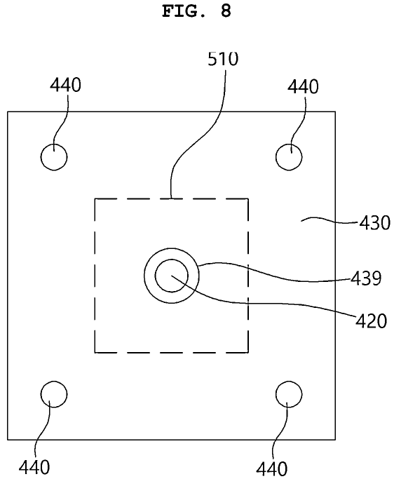

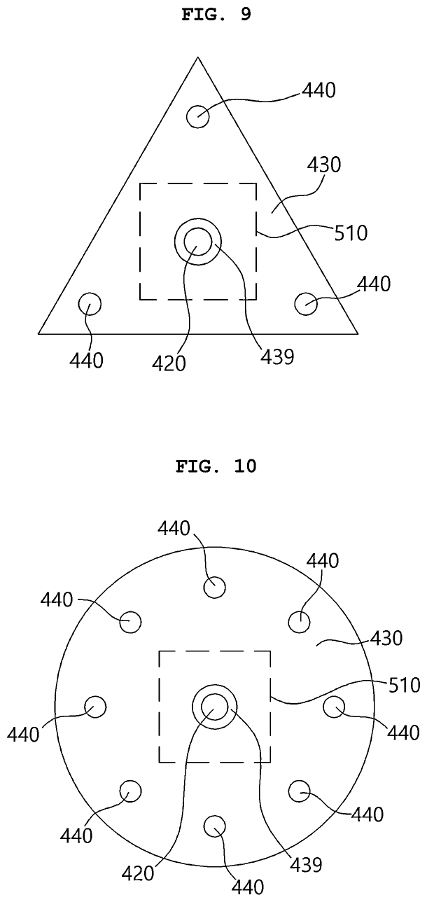

FIG. 8 is a plan view illustrating a shape of the pressing plate of the pressing unit according to an embodiment of the present invention, FIG. 9 is a plan view illustrating another shape of the pressing plate of the pressing unit according to an embodiment of the present invention, and FIG. 10 is a plan view illustrating another shape of the pressing plate of the pressing unit according to an embodiment of the present invention.

FIGS. 8 to 10 will be described by referring to FIGS. 3 to 6 in order to avoid redundant description and facilitate understanding of the description.

Referring to FIGS. 8 to 10, the pressing unit 400 may include a pressing rod 420, a pressing plate 430 and guide rods 440.

The pressing plate 430 may include a seating part 439. The seating part 439 may be disposed in an area including the center of gravity of the pressing plate 430. The pressing rod 420 may be disposed at a position corresponding to the seating part 439.

The guide rods 440 may be arranged around the pressing rod 420. The guide rods 440 may be symmetrically arranged about the pressing rod 420. The symmetrically arranged guide rods 440 may form a dispersed pressing force on the whole surface of the pressing plate 430.

Meanwhile, the lower plate 510 of the molding unit 500 may be disposed to have a smaller area than the upper plate 520. In addition, the upper plate 520 may be coupled to the pressing plate 430 in the same shape as each other.

Accordingly, the pressing force provided from the pressing plate 430 on which the dispersed pressing force is formed may be transmitted to the upper plate 520. The upper plate 520 that has received the pressing force may press the object to be molded while facing the lower plate 510.

Therefore, the pressing plate 430 having the dispersed pressing force formed on the whole surface thereof may help to form a uniform surface pressure in the molding unit 500.

In FIGS. 3 to 6, the shape of the pressing plate 430 is formed in a square shape, and the guide rods 440 may be symmetrically arranged on both sides of the pressing rod 420 about the pressing rod 420. Therefore, two guide rods 440 may be disposed to be connected to the pressing plate 430.

Referring to FIG. 8, the pressing plate 430 has the same square shape, and the guide rods 440 may be symmetrically arranged at the edge regions of the pressing plate 430 around the pressing rod 420. Therefore, four guide rods 440 may be disposed to be connected to the pressing plate 430.

As such, since the four guide rods 440 are symmetrically arranged on the pressing plate 430 about the pressing rod 420, the dispersion of the pressing force may be further improved.

Referring to FIG. 9, the pressing plate 430 may be formed in a triangular shape. The triangular-shaped pressing plate 430 may include three guide rods 440 disposed thereon. For example, when the pressing plate 430 has a triangular shape, in order to symmetrically arrange the guide rods 440 about the pressing rod 420, three guide rods 440 may be disposed on the pressing plate 430.

As such, when the pressing plate 430 has a triangular shape, since the three guide rods 440 are symmetrically arranged on the pressing plate 430 about the pressing rod 420, the dispersion of pressure may be further improved.

Referring to FIG. 10, the pressing plate 430 may be formed in a circular shape. Four to eight guide rods 440 may be disposed on the circular pressing plate 430. In this embodiment, the case where eight guide rods 440 are disposed on the pressing plate 430 is illustrated.

Specifically, when the pressing plate 430 has a circular shape, in order to symmetrically arrange the guide rods 440 about the pressing rod 420, four to eight guide rods 440 may be disposed on the pressing plate 430.

As such, when the pressing plate 430 has a circular shape, since the four to eight guide rods 440 are symmetrically arranged on the pressing plate 430 about the pressing rod 420, the dispersion of pressure may be further improved.

FIG. 11 is a flowchart illustrating a general sequence of a cartilage regeneration scaffold manufacturing method using the cartilage regeneration scaffold manufacturing apparatus according to an embodiment of the present invention, FIGS. 12 to 14 are flowcharts illustrating detailed sequences of the cartilage regeneration scaffold manufacturing method according to each step in FIG. 11, and FIGS. 15A to 20 are process diagrams illustrating the cartilage regeneration scaffold manufacturing method using the cartilage regeneration scaffold manufacturing apparatus according to an embodiment of the present invention.

FIGS. 11 to 20 will be described by referring to FIGS. 3 to 6 in order to avoid redundant description and facilitate understanding of the description.

The cartilage regeneration scaffold manufacturing method according to an embodiment of the present invention may manufacture a cartilage regeneration scaffold using the cartilage regeneration scaffold manufacturing apparatus 10.

The cartilage regeneration scaffold manufacturing apparatus 10 may include the transfer unit 300, the molding unit 500 and the pressing unit 400. The molding unit 500 may include the upper plate 520 and the lower plate 510. An object to be molded may be placed between the upper plate 520 and the lower plate 510. The cartilage regeneration scaffold manufacturing apparatus 10 may manufacture a cartilage regeneration scaffold with improved pattern uniformity on single or both sides by applying pressure to the object to be molded.

Hereinafter, the present invention will be described in more detail through examples.

### EXAMPLE

### Example 1. Fabrication of PLGA Large-Area Sheet (Single-Sided Pattern) and Measurement of Uniformity

A cartilage regeneration scaffold was manufactured using the manufacturing apparatus shown in FIG. 3.

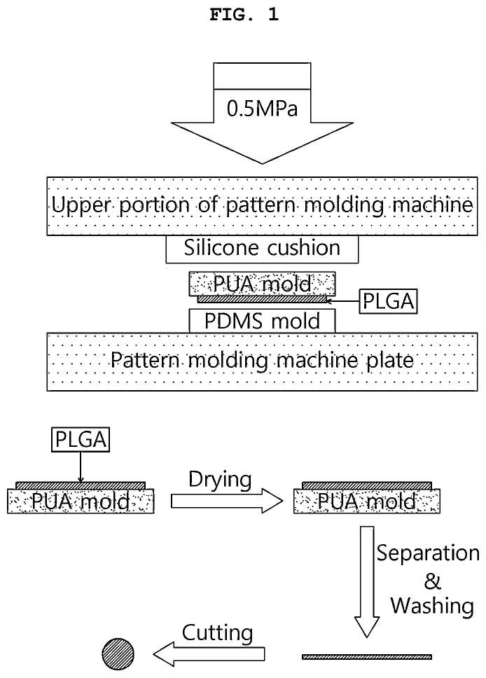

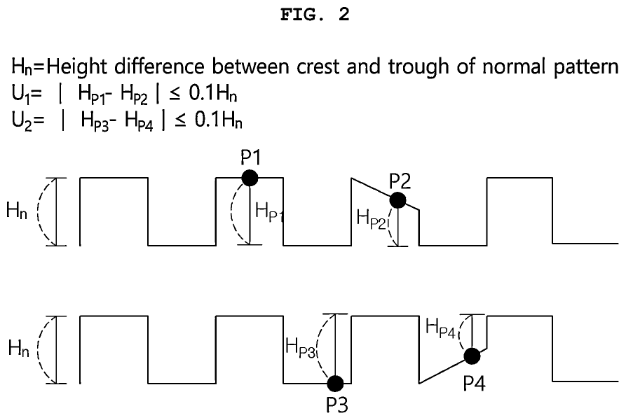

As shown in FIG. 1, a single-sided PDMS mold (flat) was laid on a plate, and 12 ml of PLGA solution (containing 10 ml of chloroform and 2.5 g of PLGA), which was prepared by dissolving 25 g of PLGA in 100 ml of chloroform, was applied evenly to a PUA mold (800 nm) in a size of 12×12 cm. Thereafter, this mold was turned over to be the PLGA solution side down and placed on a PDMS mold. Pattern molding was performed by applying pressure using the manufacturing apparatus shown in FIG. 3 for 40 minutes at room temperature.

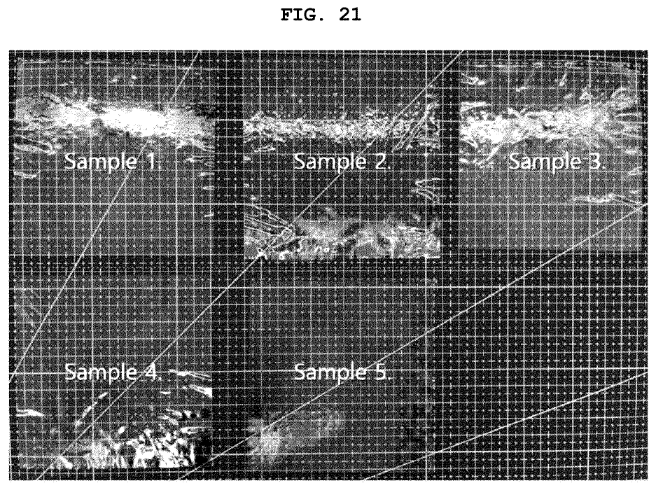

After pattern molding was completed, these molds were placed to be the solution side up on a hot plate at 70° C., and the solvent was evaporated (for about 1 hour). The hardened PLGA sheet was carefully separated from the PUA mold. The separated PLGA large-area scaffold was soaked in 70% ethanol for about 10 seconds and then washed with purified water. The washed scaffold was dried for 8 hours or more, and then cut to a size matched with the dimensions. The above process was repeated four more times to prepare a total of five large-area PLGA sheets (FIG. 21).

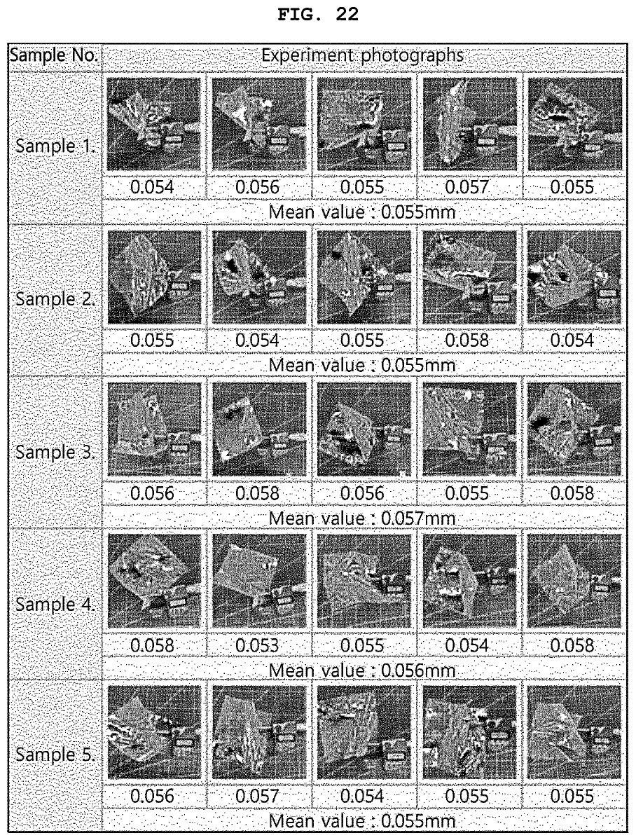

The thickness of the five prepared large-area PLGA sheets was randomly measured at five points, and an average value of each sample and an overall average value of the measured data were recorded (FIG. 22).

### Example 2. Preparation of PLGA Large-Area Sheet (Double-Sided Pattern)

A cartilage regeneration scaffold was manufactured using the manufacturing apparatus shown in FIG. 3.

As shown in FIG. 1, a double-sided PDMS mold (800 nm) was laid on a plate, and 12 ml of PLGA solution (containing 10 ml of chloroform and 2.5 g of PLGA), which was prepared by dissolving 25 g of PLGA in 100 ml of chloroform, was applied evenly to a PUA mold (800 nm) in a size of 12×12 cm. Thereafter, this mold was turned over to be the PLGA solution side down and placed on a PDMS mold. Pattern molding was continued by applying pressure using the manufacturing apparatus shown in FIG. 3 for 40 minutes after setting the temperature to 50° C.

After pattern molding was completed, the upper and lower molds were placed on a hot plate at 70° C. without separating them, and then dried for 5 hours or more while pressing with a steel plate having a weigh of 1.2 kg to prevent these molds from being separated. The hardened PLGA was carefully separated from the PDMS and PUA molds. The separated PLGA large-area scaffold was soaked in 70% ethanol for about 10 seconds and then washed three times with purified water. The washed scaffold was dried by blowing air for about 30 minutes, and then cut to a size matched with the dimensions. The above process was repeated four more times to prepare a total of five large-area PLGA sheets.

### Example 3. Measurement of Pattern Between Troughs and Crests of Large-Area PLGA Sheet Through FE-SEM Imaging

FE-SEM imaging was performed on the sheets of Examples 1 and 2 under the following conditions to measure the pattern distance between the troughs and crests, and pattern uniformity of nanopattern.
Resolution: 0.6 nm
Magnification: ×20 to 2,000,000
EDS detector (ATW2, 127 eV, 50 mm²)

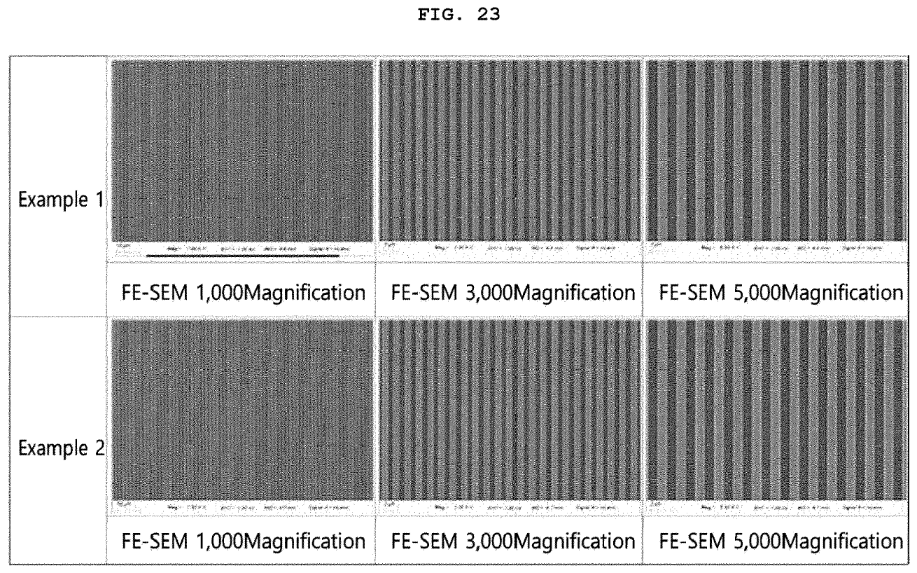

As a result, the distance between the crest and the trough of each pattern in the large-area sheets of Examples 1 and 2 was 790 nm, and a 1:1 ratio of the crest to the trough was maintained (FIG. 23). In addition, a deviation between five random points on the crest and five random points on the trough of the nanopattern was less than 10 nm, such that there was no substantial difference.

What is claimed is:

1. A cartilage regeneration scaffold comprising a biocompatible polymer and having a pattern for cartilage regeneration formed on single or both sides,
wherein the pattern comprises repeating crests and troughs, and
an area of the pattern, which satisfies a uniformity (U) of Equation 1 below, is 95% or more of a total area of the pattern:
$$U = \vert {H}_{P 1} - {H}_{P 2} \vert \leq 0.1 {H}_{n} \qquad [ Equation 1 ]$$
wherein H_{P1} refers to a height at P1, H_{P2} refers to a height at P2, and Hₙ refers to a height difference between the crest and the trough in a normal pattern.

2. The cartilage regeneration scaffold according to claim 1, wherein both P1 and P2 are points located on the crest or on the trough.

3. The cartilage regeneration scaffold according to claim 1, wherein the area which satisfies a uniformity of Equation 1 is 99% or more of the total area of the pattern.

4. The cartilage regeneration scaffold according to claim 1, wherein the scaffold has a thickness of 30 to 100 μm.

5. The cartilage regeneration scaffold according to claim 1, wherein the biocompatible polymer is any one selected from the group consisting of polycaprolactone, polylactide-co-glycolide, polyethylene glycol, polyethylene oxide, polylactic acid and polyglycolic acid.

6. The cartilage regeneration scaffold according to claim 1, wherein the biocompatible polymer is polylactide-co-glycolide including 65 to 85 mol % of lactide and 15 to 35 mol % of glycolide.

7. The cartilage regeneration scaffold according to claim 1, wherein the pattern has a straight line or curved line shape in which the crests and the troughs are repeated side by side.

8. The cartilage regeneration scaffold according to claim 1, the single or both sides is/are applied with any one selected from the group consisting of collagens, growth factors, stem cells, exosomes and therapeutic drugs.

9. The cartilage regeneration scaffold according to claim 1, wherein fibrin is applied to an edge of the scaffold.

10. A method for manufacturing the cartilage regeneration scaffold according to claim 1, the method comprising:
applying a polymer solution containing a biocompatible polymer to a polyurethane acrylate mold to prepare a sheet-like semi-cured product;
applying pressure to the semi-cured product with a polydimethylsiloxane mold to prepare a patterned semi-cured product; and
drying the patterned semi-cured product.

11. The method according to claim 10, wherein the patterned semi-cured product is prepared by placing the semi-cured product between the polyurethane acrylate mold and the polydimethylsiloxane mold, and then applying pressure thereto with upper and lower plates.

12. The method according to claim 10, wherein the polyurethane acrylate mold or the polydimethylsiloxane mold has concavo-convex portions formed thereon, for forming the pattern.

13. The method according to claim 11, wherein the upper plate comprises a pressing rod and a guide rod.

14. The method according to claim 10, wherein the pressure is 0.40 to 0.70 Pa.
